(19) United States
(12) Patent Application Publication
(10) Pub. No.: US 2003/0134803 A1
(43) Pub. Date: Jul. 17, 2003

(54) Use of sulfonated compounds as a barrier contraceptive

(75) Inventors: Gary N. Cherr, Penngrove, CA (US); Edward R. Salinas, Vallejo, CA (US)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA

(21) Appl. No.: 10/076,902

(22) Filed: Feb. 13, 2002

Related U.S. Application Data
(60) Provisional application No. 60/349,144, filed on Jan. 15, 2002.

(51) Int. Cl. A61K 31/70; A61K 31/79; A61K 31/655; A61K 31/409; A61K 31/185

(52) U.S. Cl. 514/22; 424/78.25; 514/150; 514/410; 514/553

(57) ABSTRACT

This invention provides methods, compositions and contraceptive devices that use sulfonated compounds that interact with sperm to inhibit fertilization. Natural contraceptive methods, compositions and contraceptive devices are also included. These natural contraceptives use sulfonated compounds isolated from natural sources. Methods, compositions and contraceptive devices are also provided that use a lignin and/or a derivative thereof.

### CROSS-REFERENCE TO RELATED APPLICATIONS

This application is related to U.S. S No. 60/349,144 “THE USE OF SULFONATED COMPOUNDS AS A BARRIER CONTRACEPTIVE” by Cherr et al., filed Jan. 15, 2002, the disclosure of which is incorporated herein by reference. The present application claims priority to and the benefit of this prior application, pursuant to 35 U.S.C. §119(e).

### STATEMENT AS TO RIGHTS TO INVENTIONS MADE UNDER FEDERALLY SPONSORED RESEARCH AND DEVELOPMENT

This work is support from a grant from the National Institutes of Health to Principal Investigator P. Primakoff. The government may have certain rights in this invention.

### FIELD OF THE INVENTION

This invention relates to methods, compositions and devices involved in inhibiting fertilization. Fertilization is inhibited in the present invention by using compounds that interact with sperm. The interaction of the compounds with the sperm can prevent the sperm from interacting with the zona pellucida. The compounds of the present invention include sulfonated compounds, sulfonated compounds from natural sources, lignins and derivatives of lignins.

### BACKGROUND OF THE INVENTION

The fertilization of an oocyte is a complex process. One region of the oocyte involved in fertilization is the zona pellucida (ZP). The ZP is a covering that surrounds mammalian oocytes. The ZP is formed during the development of the oocyte and follicular cell differentiation. The ZP serves to protect the oocyte and embryo until implantation in the uterine wall and serves as an attachment site for sperm. Upon attachment of the sperm to the zona pellucida and penetration of the zona pellucida by the sperm, the oocyte becomes fertilized. ZP may also prevent polyspermy because fertilization of the oocyte alters sperm binding to the ZP.

A process that prepares the sperm for fertilization of the oocyte is capacitation. Capacitation is a period of conditioning in the female reproductive tract. During capacitation, the ejaculated sperm remain in the female reproductive tract and undergo changes to the sperm cell membrane to prepare sperm for binding to the oocyte. See, e.g., Gilbert, Developmental Biology (2^{nd} edition) (1988) Sinauer Associates, Inc. (Sunderland, Mass.).

Various compounds and methods have been used to prevent or disrupt fertilization by acting upon a certain step in the process, e.g., by changing the oocyte, by changing the development of the oocyte, by changing progression through fertilization, and/or by acting upon the sperm. Contraceptives are compounds and/or devices that alter the fertilization process. For example, barrier methods are one means of providing an effective vaginal contraceptive. These methods can be improved by supplying or designing barriers with compounds that interfere with the fertilization process.

For example, spermicides, such as nonoxynol-9, work by immobilizing spermatozoa (e.g., spermicides have spermicidal activity). Nonoxynol-9 is a nonionic detergent that nonspecifically disrupts cell membranes. The properties that make nonoxynol-9 a potent cytotoxic agent to sperm can also be cytotoxic to vaginal epithelial and to normal vaginal flora, which can result in an increased risk of sexually transmitted diseases or other infections. See, e.g., Maguire et al., Comparison of microbicides for efficacy in protecting mice against vaginal challenge with herpes simplex virus type 2, cytotoxicity, antibacterial properties, and sperm immobilization, (2001) Sexually Transmitted Diseases, 28(5):259-265.

While the majority of barrier contraceptives have focused on the cytotoxic effects on sperm, it would be beneficial to have a contraceptive that targets specific sperm surface molecules that are involved in fertilization events, yet are not cytotoxic to vaginal epithelia and do not upset local flora. This has been challenging, because freshly ejaculated sperm have to undergo numerous changes during capacitation as well as transport in the female tract, and thus would typically not be affected by vaginal contraceptives that are not directly spermicidal. It is also desirable to have a contraceptive that works with a relatively short exposure to the sperm while still maintaining its effectiveness after capacitational changes, e.g., removal of cell surface molecules, has occurred.

The present invention uses sulfonated compounds to inhibit fertilization. Methods, compositions and contraceptive devices to achieve these goals are provided. A fuller understanding of the invention will be provided by review of the following.

### SUMMARY OF THE INVENTION

Methods, compositions and contraceptive devices are provided that use sulfonated compound to inhibit fertilization. The sulfonated compounds include, e.g., lignosulfonic acid, polyanetholesulfonic acid, polyvinylsulfonic acid, poly(2-acrylamido-2-methyl-1-propanesulfonic acid), poly(2-acrylamido-2-methyl-1-propanesulfonic acid co-acrylonitrile), poly(2-acrylamido-2-methyl-1-propanesulfonic acid-co-styrene), poly(4-vinylpyridinium p-toluenesulfonate), sulfonic acid azo dye, sulfonic acid derivative of a porphyrin, sulfonic acid derivative of triphenylmethane, sulfonic acid derivative of stilbene, sulfonated phenylpropane, sulfonated kraft lignin, and derivatives thereof. Sulfonated compounds can also be derived from a natural source. Other compounds of the invention include lignins and derivatives thereof.

The advantages of these contraceptives over other contraceptives are that they are not cytotoxic and that they are effective before, during and after capacitational changes. In addition, a relatively short exposure of the compound to the sperm will block the ability of the treated sperm to fertilize an oocyte.

Accordingly, the methods comprise administering an effective amount of a compound to an animal, the compound comprising at least one sulfonated compound, wherein the compound interacts with sperm and wherein the at least one sulfonated compound is selected from the group consisting of: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, and derivatives thereof. Optionally, the compound is a polysulfonated compound. Typically, the compound is in an aqueous solution.

In another embodiment of the methods, the interaction of the compound and sperm occurs at the sperm surface, or optionally, at head of the sperm. In one embodiment, the interaction includes the compound binding to the sperm. In another aspect of the methods, the compound inhibits the sperm interaction with a zona pellucida.

The methods of the present invention can further comprise treating the sperm with the compound for, e.g., at least about 3 minutes, e.g., at least about 5 minutes, e.g., at least about 10 minutes; e.g., at least about 20 minutes, e.g., at least about 30 minutes; e.g., at least about 40 minutes; or e.g., at least about 60 minutes.

Typically, the treatment occurs at about 37° C., e.g., body temperature. Optionally, the treatment can occur at about room temperature or another temperature relevant to the organism or animal at issue, such as a normal oceanic temperature. The sperm can optionally be treated after ejaculation, or optionally, at various times after ejaculation, e.g., after about 15 minutes, e.g., after about 30 minutes, e.g., after about 1 hour, e.g., after about 2 hours, e.g., after about 4 hours, e.g., after about 6 hours, e.g., after about 12 hours, e.g., after about 24 hours, or, e.g., a time prior to the sperm interacting with the zona pellucida.

In one aspect, the methods of the present invention include the administration of the compound after ejaculation. Optionally, the compound can be administered prior to ejaculation. In further aspects of the methods, the compound is administered vaginally.

In another embodiment of the methods, the compound is in a pharmaceutically acceptable formulation. The formulation can be, e.g., one of the following: a foam, a cream, a gel, a jelly, a douche, an aerosol, a film, a tablet, a sponge, a vaginal suppository, an impregnated tampon, a controlled delivery device, a vaginal ring, an intrauterine device, a lubricant on a male condom, a lubricant on a female condom, a lubricant on a cervical cap, a lubricant on a cap diaphragm, or the like.

Typically, the animal on which the methods of the present invention are used is a primate, but other animals are also included. For example, such animals include any or all mammals, e.g., humans, canines, felines, rodents and many others. Indeed, the methods are broadly useful, having shown activity on animals as disparate as sea urchin and mammals.

The methods of the present invention also comprise administering an effective amount a compound derived from a natural source to an animal, the compound comprising at least one sulfonated compound, e.g., a lignosulfonic acid (LSA), wherein the at least one sulfonated compound interacts with sperm. The natural source of the methods of the present invention can be, e.g., a lignin, a plant, a fungus, an algae, and/or the like. In one embodiment, the compound can be obtained from a source that includes genetically altered organisms. Additional aspects of these methods include those embodiments described above.

Another method comprises administering an effective amount of a compound to an animal, the compound comprising at least one lignin and/or a derivative thereof, wherein the at least lignin and/or derivative thereof interacts with sperm. In further embodiments, the compound is sulfated and/or sulfonated. These methods also include the previously described embodiments.

Another embodiment of the methods includes administering an effective amount of a compound to an animal, the compound comprising at least one sulfonated compound, wherein the compound interacts with sperm and wherein the at least one sulfonated compound is other than a polystyrene sulfonate, a long chain alkyl sulfonate, a long chain alkenyl sulfonate, a sulfoalkyl alkanoate salt, a sodium tetradecyl sulfonate, a sulfonated hesperidin, a substituted benzenesulfonic acid formaldehyde co-polymer, a H₂SO₄-modified mandelic acid, a condensation polymer product produced a condensation reaction of an aromatic sulfonic acid and an aldehyde, a formaldehyde naphthalenesulfonic acid condensation polymer, a 8-anilino-1-naphthalenesulfonate, a N-(6 aminohexyl)-5-chloro-1-naphthalenesulfonamide, a N-(6 aminohexyl)-5-chloro-2-naphthalenesulfonamide, and a N-(6 aminohexyl)-5-bromo-2-naphthalenesulfonamide. These methods also include the previously described embodiments.

Compositions are also included in the present invention, e.g., those noted above in the context of the methods of the invention. For example, compositions of the invention include, e.g., at least one sulfonated compound, a pharmaceutically acceptable excipient and a sperm. Other compositions include at least one sulfonated compound and a spermicide, e.g. Nonoxynol 9™. Typically, the at least one sulfonated compound is selected from the group consisting of: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, and derivatives thereof. In one aspect, the compound is a polysulfonated compound.

Compositions also include a compound isolated from a natural source, a pharmaceutically acceptable excipient and a sperm, where the compound interacts with sperm and where the compound comprises at least one sulfonated compound, e.g., a lignosulfonic acid (LSA). Another composition comprises a compound isolated from a natural source and a spermicide, e.g. Nonoxynol 9™, where the compound comprises at least one sulfonated compound, e.g., a lignosulfonic acid (LSA). The natural sources of such compounds can be e.g., a lignin, a plant, a fungus, an algae, and/or the like. Optionally, the compounds can be obtained from a source that includes, e.g., genetically altered organisms. Compounds in these compositions can optionally be polysulfonated compounds.

Other compositions of the present invention include a composition comprising a lignin and/or a derivative thereof, a pharmaceutically acceptable excipient and a sperm. Compositions also include a composition comprising a lignin and/or a derivative thereof and a spermicide, e.g. Nonoxynol 9™. Optionally, these compositions are sulfated and/or sulfonated.

Devices are also aspects of the present invention, e.g., those which are useful in the methods of the invention, and/or which incorporate the compositions herein. For example, contraceptive devices of the present invention can include a contraceptive device and at least one sulfonated compound, e.g., in a formulation, e.g., where the device is a sponge, a tampon, an intrauterine device, a vagina ring, a male condom, a female condom, a cervical cap, a diaphragm and the like and where the formulation is one or more of the following: a foam, a cream, a gel, a jelly, a douche, an aerosol, a film, a suppository, a tablet or the like. Typically, the least one sulfonated compound is one of the following: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, or one or more derivatives thereof.

A contraceptive device can also include a device and a compound isolated from a natural source, where the compound in such a device comprises at least one sulfonated compound. Examples of devices and formulations are described above. Typically, the sulfonated compound is a lignosulfonic acid (LSA). The natural source of the compound in the contraceptive device can be, e.g., a lignin, a plant, a fungus, an algae, and/or the like. Optionally, the isolated compound can also be obtained from genetically altered organisms.

Other contraceptive devices of the present invention include a contraceptive device, as described above, and a compound comprising at least one lignin and/or a derivative thereof in a formulation, as described above. The compound in the contraceptive device can optionally be sulfated and/or sulfonated.

### BRIEF DESCRIPTION OF THE DRAWINGS

FIG. 1 graphically illustrates the reduced ability of LSA-treated capacitated sperm to bind to the zona pellucida of immature oocytes by comparing control sperm, sperm treated with 1.5 mg/ml LSA either before centrifugation through Percoll (pre-capacitation; Pre-C) or after activation (post capacitation; Post C). Columns represent averages of sperm bound per zona with sperm from 4 different males and a total of 8 zona per treatment. Error bars represent the standard error of means. Different letters above columns indicate significant differences between treatment means (p>0.001).

FIG. 2 illustrates that the motility and progression of sperm are not significantly altered in sperm treated with LSA in zona pellucida binding assays (ZBA) and in vitro fertilization (IVF).

FIG. 3 illustrates the reduced ability of PASA-treated or LSA-treated sperm, both post-capacitation (PC) or pre-wash (PW), to bind to the zona pellucida of immature oocytes compared to control.

FIG. 4 illustrates the effect of fucoidan treated sperm to interact with the zona pellucida by comparing control sperm, and sperm treated with 1.5 mg/ml Fucoidin either before centrifugation through Percoll (pre-capacitation; Pre-C) or after activation (post capacitation; Post-C). Columns represent averages of sperm bound per zona with sperm from 4 different males and a total of 8 zona per treatment. Error bars represent the standard error of means. Different levels above columns indicate significant differences between treatment means (p>0.001).

FIG. 5 illustrates the reduction in fertilization rates of sperm treated with LSA in IVF trials using both pre-wash and post-capacitation LSA-treated sperm compared to controls.

FIG. 6, Panels A, B and C illustrate sperm-zona binding studies. Panel A illustrates sperm-zona binding with control sperm, while Panel B and Panel C illustrate sperm-zona binding with LSA-treated sperm pre-capacitation and post-capacitation, respectively.

FIG. 7, Panels A, B, C and D illustrate the localization of sperm labeled with FITC-LSA both pre-, e.g., Panels A and B, and post-capacitation, Panels C and D. Panels A and C illustrate fluorescence micrographs, while Panels B and D illustrate the corresponding phase contrast images.

### DETAILED DESCRIPTION

The methods, compositions and devices of the present invention are directed to sulfonated compounds, which interact with sperm to inhibit or prevent fertilization. These compounds can be isolated from natural sources to provide a natural non-toxic contraceptive. Other embodiments include the use of lignin and derivatives thereof as a contraceptive.

Methods

The contraceptive methods of the present invention include administering compounds that interact with sperm and inhibit fertilization. In one embodiment, the methods comprise administering an effective amount of a compound to an animal, the compound comprising at least one sulfonated compound, wherein the compound interacts with sperm. In another embodiment of the methods, at least one sulfonated compound, e.g., a lignosulfonic acid, is derived from a natural source, e.g., from a lignin, e.g., from a plant, e.g., from a fungus, e.g., from an algae, and the like. Optionally, the at least one sulfonated compound is obtained from a genetically altered organism.

One class of methods comprises administering an effective amount of a compound to an animal, the compound comprising at least one lignin and/or a derivative thereof, wherein the compound interacts with sperm. In further embodiment, the compound is sulfated and/or sulfonated.

In another embodiment of the methods include administering an effective amount of a compound to an animal, the compound comprising at least one sulfonated compound, wherein the compound interacts with sperm and wherein the at least one sulfonated compound is other than a polystyrene sulfonate, a long chain alkyl sulfonate, a long chain alkenyl sulfonate, a sulfoalkyl alkanoate salt, a sodium tetradecyl sulfonate, a sulfonated hesperidin, a substituted benzenesulfonic acid formaldehyde co-polymer, a H₂SO₄-modified mandelic acid, a condensation polymer product produced a condensation reaction of an aromatic sulfonic acid and an aldehyde as described in U.S. Pat. No. 5,958,399 to Sonderfan et al., entitled “Sulfonic Acid and Aldehyde Condensation Polymers for Preventing Pregnancy” issued on Sep. 28, 1999, a formaldehyde naphthalenesulfonic acid condensation polymer, a 8-anilino-1-naphthalenesulfonate, a N-(6 aminohexyl)-5-chloro-1-naphthalenesulfonamide, a N-(6 aminohexyl)-5-chloro-2-naphthalenesulfonamide, and a N-(6 aminohexyl)-5-bromo-2-naphthalenesulfonamide.

Typically, the animal for which fertilization is to be blocked is a primate, but other animals are also targets for inhibiting fertilization. For example, an animal can be a mammal, a human, a canine, a feline, a rodent, a vertebrate, a non-mammal, an insect, a fish, an invertebrate or the like. Indeed, the methods are broadly useful, having shown activity on animals as disparate as sea urchins and mammals.

The interaction of the compound and the sperm typically occurs, e.g., at the surface of the sperm; or, e.g., at head of the sperm. For example, LSA can compete for the natural sulfated ligand, egg jelly, on the sperm surface. See, e.g., Salinas and Cherr, Identification of cell surface domains for lignosulfonic acids derived from pulp mill effluent, In Abstracts, 21^{st} Annual Meeting for the Society of Environmental Toxicology and Chemistry, Nashville, Tenn., November 2000, Society of Environmental Toxicology and Chemistry, Pensacola, Fla. In one aspect, the interaction includes the compound binding to the sperm. In another aspect, the methods include where the compound inhibits the sperm interaction with a zona pellucida.

An effective amount of the compound is an amount sufficient to inhibit fertilization but not kill sperm. Typically, an effective amount is a concentration, e.g., between about 0.1 mg/ml and about less than 18 mg/ml, e.g., between about 0.25 mg/ml and about 15 mg/ml, e.g., between about 0.5 mg/ml and about 12 mg/ml, e.g., between about 1.0 mg/ml and about 10 mg/ml, e.g., between about 0.5 mg/ml and about 5.0 mg/ml, e.g., between about 1.0 mg/ml and about 2.5 mg/ml, or e.g., about 1.5 mg/ml.

The methods of the present invention can further comprise treating the sperm with an effective amount of the compound for, e.g., at least about 3 minutes, e.g., at least about 5 minutes, e.g., at least about 10 minutes; e.g., at least about 20 minutes, e.g., at least about 30 minutes; e.g., at least about 40 minutes; or e.g., at least about 60 minutes. Typically, the treatment occurs at about 37° C., e.g., body temperature. Optionally, the treatment can occur at about room temperature or another temperature relevant to the organism or animal at issue, such as a normal oceanic temperature. The sperm can optionally be treated after ejaculation, or optionally, various times after ejaculation, e.g., after about 15 minutes, e.g., after about 30 minutes, e.g., after about 1 hour, e.g., after about 2 hours, e.g., after about 4 hours, e.g., after about 6 hours, or, e.g., after about 12 hours, e.g., after about 24 hours, or, e.g., a time prior to the sperm interacting with the zona pellucida.

The administration of the compounds of the present invention can occur after ejaculation, or optionally, prior to ejaculation. Optionally, the compounds of the present invention can be reapplied after completion of sexual activity, or optionally after completion of a first sexual activity, a second sexual activity, etc. The administration of the compounds involved in this invention can be accomplished in a variety of ways, e.g., the compound can be administered vaginally. In another embodiment of the methods, the compound is in a pharmaceutically acceptable formulation, which can be optionally used to administer the compound. For example, a formulation can be, e.g., one of the following: a foam, a cream, a gel, a jelly, a douche, an aerosol, a film, a tablet, a sponge, a vaginal suppository, an impregnated tampon, a controlled delivery device, a vaginal ring, an intrauterine device, a lubricant on a male condom, a lubricant on a female condom, a lubricant on a cervical cap, a lubricant on a cap diaphragm and/or the like.

Other types of administration are also included in the present invention. For example, the administration of the compounds can occur in vitro or ex vivo. Compounds can also be administered into the surrounding, e.g., sea water, fresh water, sand, dirt or the like, to treat animals, e.g., such as non-mammals, e.g., amphibians, e.g., reptiles, or e.g., fish.

Compositions

Compositions are also included in the present invention. Compositions include compounds of the present invention with sperm and a pharmaceutically acceptable excipient. For example, a composition can comprise at least one sulfonated compound, a pharmaceutically acceptable excipient and a sperm.

Other compositions include a compound isolated from a natural source, a pharmaceutically acceptable excipient and a sperm, wherein the compound interacts with the sperm and wherein the compound comprises at least one sulfonated compound, e.g., a lignosulfonic acid (LSA). The natural sources of such compounds can be e.g., a lignin, a plant, a fungus, an algae, and/or the like. Optionally, the compound can be obtained from a genetically altered organism.

In another embodiment, compositions of the present invention include a composition comprising a lignin and/or a derivative thereof, a pharmaceutically acceptable excipient and a sperm. Optionally, the compound is sulfated and/or sulfonated.

The compounds of the present invention can allow the sperm to remain mobile while preventing or inhibiting fertilization of the oocyte by the treated sperm. The compositions described above can be used in a variety of ways, e.g., the compositions can be used in experiments and/or kits as controls and as comparisons, etc., where it is required and/or desired that sperm be mobile, yet have lost or diminished capacity to fertilize an oocyte. Such controls are useful e.g., for the development of new contraceptives, determining the timing of treatment, the cytotoxity of the treatment, and/or the like.

Compounds described in the present invention can also be present with a spermicide, e.g., Nonoxynol 9™. For example, a composition can comprise at least one sulfonated compound and a spermicide. In another embodiment, the composition comprises a compound isolated from a natural source and a spermicide, where the compound comprises at least one sulfonated compound, e.g., a lignosulfonic acid (LSA). In another aspect, the composition comprises a compound obtained from a genetically altered organism and a spermicide, where the compound comprises at least one sulfonated compound, e.g., a lignosulfonic acid (LSA). Compositions can also include a composition comprising a lignin and/or a derivative thereof and a spermicide. Optionally, this composition is sulfated and/or sulfonated.

Devices

Contraceptive devices comprising a device and a compound of the present invention, in a formulation are also included in the present invention. Devices include, e.g., a sponge, e.g., a tampon, e.g., an intrauterine device, e.g., a vagina ring, e.g., a male condom, e.g., a female condom, e.g., a cervical cap, e.g., a diaphragm, or the like. Devices, e.g., vaginal rings, are further described in U.S. Pat. No. 3,545,439 to Duncan, entitled “Medicated Devices and Methods” issued Dec. 8, 1970; U.S. Pat. No. 3,920,805 to Roseman, entitled “Pharmaceutical Devices and Method” issued Nov. 18, 1975; U.S. Pat. No. 4,012,496 to Schopflin et al., entitled “Vaginal Ring” issued Mar. 15, 1977; U.S. Pat. No. 4,012,497 to Schopflin, entitled “Drug excipient of silicone rubber” issued Mar. 15, 1977; U.S. Pat. No. 4,237,885 to Wong et al., entitled “Delivery system with mated members for storing and releasing a plurality of beneficial agents” issued Dec. 9, 1980; U.S. Pat. No. 4,286,587 to Wong et al., entitled “Vaginal drug delivery system made from polymer” issued Sep. 1, 1981; and U.S. Pat. No. 4,292,965 to Nash et al., entitled “Intravaginal ring” issued Oct. 6, 1981. The formulation, which can be, e.g., throughout, e.g., within, or e.g., applied on the interior and/or exterior surfaces of the device, include, e.g., a foam, a cream, a gel, a jelly, a douche, an aerosol, a film, a suppository, a tablet, a lotion, a liniment, a salve, an oil, and/or the like.

Compounds used with such devices typically include at least one sulfonated compound. The compound is optionally isolated from a natural source, which contains at least one sulfonated compound, e.g., a lignosulfonic acid (LSA). The natural source of the compound can be, e.g., a lignin, a plant, a fungus, an algae, and/or the like. Optionally, the compound is obtained from a genetically altered organism. In another embodiment, the compound with the device comprises at least one lignin and/or a derivative thereof. The compound can optionally be sulfated and/or sulfonated.

Sulfonated Compounds

Sulfonated compounds are compounds that contain at least one sulfonate group. Various sulfonated compounds can be used in the present invention, e.g., lignosulfonic acid (LSA), polyanetholesulfonic acid (PASA), other compounds described herein, derivatives thereof, and the like.

LSA is a highly sulfonated macromolecule ranging in molecular weight from 5 kilodaltons to several hundred kilodaltons and composed of sulfonated phenylpropane monomers, and derivatives thereof. LSA can be composed of homologous and heterologous repeating units of sulfonated phenylpropane and derivatives thereof, e.g., units of substituted sulfonated guaiacylpropane, substituted sulfonated syringylpropane, substituted sulfonated hydroxyphenylpropane, and the like. This is further described in the following publications and references cited within: Loomis and Beyer, Heparin-like anticoagulant action of sulfonated lignins from commercial waste sulfite liquor (1953), J. Pharmacol. Exp. Ther, 109:21; Vocac J A, and Alphin R S, Effects and mechanism of action of a lignosulphonate on experimental gastric ulceration in rats (1968) Eur J Pharmacol, 4:99-102; and, Pearl, The Chemistry of Lignin (1967) Edward Arnold, Marcel Dekker, London, New York. LSA is also known by other names, e.g., ligninsulfonate, ligninsulfonic acid, lignonsulfonate, ligninsulfate, LST 7, poly (lignosulfonic acid), protectol W, sulfite lignin along with others, which are all included in the present invention.

Other sulfonated compounds can also be used in the present invention. They include e.g., a polyvinylsulfonic acid, e.g., a poly(2-acrylamido-2-methyl-1-propanesulfonic acid), e.g., a poly(2-acrylamido-2-methyl-1-propanesulfonic acid co-acrylonitrile), e.g., a poly(2-acrylamido-2-methyl-1-propanesulfonic acid-co-styrene), e.g., a poly(4-vinylpyridinium p-toluenesulfonate), e.g., a sulfonic acid azo dye (e.g., Evans Blue, e.g., Chicago Sky Blue, e.g., Direct Yellow 50, e.g., Congo Red), e.g., a sulfonic acid derivative of a porphyrin, e.g., a sulfonic acid derivative of a triphenylmethane, e.g., a sulfonic acid derivative of a stilbene, e.g., a sulfonated phenylpropane, e.g., a sulfonated kraft lignin, e.g., polysulfonated compounds that exhibit microbicidal activity, e.g., macromolecular polysulfonated compounds, and e.g., derivatives thereof. Others compounds of the present invention include, e.g., polyanionic macromolecular compounds, e.g., carboxylated compounds that show anti-microbial activity, and the like.

In some embodiments, the compound of the present invention comprises a polysulfonated compound. Various derivatives, e.g., salt forms, e.g., glycosylated forms, e.g., (poly)saccharide forms and the like, of the above compounds are also included in the present invention. Salts derivatives of the compounds can include, e.g., calcium, e.g., sodium, e.g., ammonium, e.g., chromium, e.g., magnesium, and the like. For example, salts derivatives of compounds include but are not limited to, e.g., lignosulfonic acid, sodium salt, lignosulfonic acid, calcium salt, poly(vinylsulfonic acid, sodium salt) and the like. Polymers of the above compounds are also included in the present invention. The polymers can comprise homologous monomer units or heterologous monomer units.

Most of the compounds of the present invention are commercially available, e.g., from Aldrich Chemical (St. Louis, Mo.). Alternatively, one skilled in the art can isolate these compounds based on publications and procedures known by one skilled in the art. These compounds are “isolated” when they are partially or completely separated from components with which it is normally associated (cells, organelles, proteins, nucleic acid, etc.), e.g., in the organism or cell from which it was derived, or when they are recovered from a natural source or a natural environment. Some of the publications and procedures are described in more detail below. For example, LSA can be isolated from, e.g., a pulp mill process, e.g., sulfite mill, or e.g., an alkaline Kraft Mill. This is further described in the following publications and references cited within: U.S. Pat. No. 4,935,239 to Machida et al., entitled “Composition for Antiviral Medicines” issued Jun. 19, 1990; U.S. Pat. No. 2,838,483 to Jentzen, entitled “Method of Separating Lignosulfonic Acids” issued Jun. 10, 1956, McCubbins N. State-of-the-art of the pulp and paper industry and its environmental protection practices. Environmental Protection Services (EPS) Economic and Technical Review Report xviii 1983; 119-128; Kringstad and Lindstrom, Spent liquors from pulp bleaching (1984) Environ Sci Technol., 18:236A-248A; and, Higashi R M, et al., A Polar High Molecular Mass Constituent of Bleached Kraft Mill Effluent Is Toxic To Marine Organisms (1992b) Environmental Science & Technology, 26:2413-2420.

Typically, the compound can be formulated in an aqueous solution, e.g., water, e.g., sterile water, e.g., saline sterile water, e.g., a Ringer's solution, or e.g., an isotonic sodium chloride solution. Optionally, the compound can be formulated in a non-aqueous form, e.g., as dried tablet, or e.g., a powder, to use, e.g., directly, or e.g., after rehydrating. The compound can also be present with other ingredients, e.g., pH modifiers, e.g., stabilizers, e.g., buffers, e.g., surfactants, e.g., moisturizers, e.g., colorants, e.g., thickeners, e.g., scents, e.g., flavorings, e.g., fragrances, e.g., perfumes, and the like. In some embodiments, the compound is found with a pharmaceutically acceptable excipient. Pharmaceutically acceptable excipients or carriers can include a wide variety of suitable compounds of pharmaceutical compositions. Typically, a pharmaceutically acceptable excipient is chosen based in part by the particular composition of the compound(s) of the present invention, as well as by the particular method used to administer the compound(s) of the present invention. For example, a pharmaceutical acceptable carrier includes an aqueous solution, a non-aqueous form, and a formulation, as described above and below, and the like.

In some embodiments of the present invention, the compound is in a formulation, e.g., a foam, a cream, a gel, a jelly, a douche, an aerosol, a film, a tablet, a sponge, a vaginal suppository, an impregnated tampon, a controlled delivery device, a vaginal ring, an intrauterine device, a lubricant on a male condom, a lubricant on a female condom, a lubricant on a cervical cap, a lubricant on a cap diaphragm, and/or the like.

Sulfonated compounds, e.g., LSA, are desirable as a contraceptive because they are effective as a contraceptive with fresh non-capacitated sperm and they lack cytotoxicity, which is described in more detail below. For example, formulations containing LSA prevent infection with sexually transmitted Herpes Simplex virus. This is further described in the following publication and references found within: U.S. Pat. No. 4,185,097 to Ward and Tankersley, entitled “Method of combating Herpes simplex viruses with lignosulfonates” issued Jan. 22, 1980. See also Maguire et al., 2001, supra, which describes that other polyanionic macromolecules, both sulfated and sulfonated, prevent sexually transmitted diseases when used in vaginal applications. Thus, sulfonated compounds can be capable of functioning both as a contraceptive and as a microbicide.

As mentioned above, sulfonated compounds can have various biological activities. For example, sulfonated compounds are used as antivirals, antimicrobials, and, e.g., in the case of polystyrene sulfonates, sulfonic acid derivatives of naphthalene and long chain alkyl and alkenyl sulfonates, as contraceptives. Publications and references cited within describing these activities include: Anderson et al., Evaluation of Poly(Styrene-4-Sulfonate) as a Preventive Agent for Conception and Sexually Transmitted Diseases, (2000) J. of Andrology, 21:862-875; Herold, et al., Poly (Sodium 4-Styrene Sulfonate): An Effective Candidate Topical Antimicrobial for the Prevention of Sexually Transmitted Diseases, (2000), JID, 181: 770-773; U.S. Pat. No. 6,239,182 to Zaneveld et al., entitled “Method for Preventing Sexually Ttransmitted Diseases” issued May 29, 2001; U.S. Pat. No. 5,958,399 to Sonderfan et al., entitled “Sulfonic Acid and Aldehyde Condensation Polymers for Preventing Pregnancy” issued Sep. 28, 1999; U.S. Pat. No. 4,493,699 to Zimmerman et al., entitled “Contraceptive Methods” issued Jan. 15, 1985; U.S. Pat. No. 4,469,671 to Zimmerman et al., entitled “Contraceptive Device” issued Sep. 4, 1984; U.S. Pat. No. 4,432,967 to Szymanski entitled “Contraceptive Composition” issued Feb. 21, 1984; U.S. Pat. No. 4,419,366 to Cormier entitled “Use of N-(6 AminoHexyl)-5-Chloro-1-Naphthalenesulfonamide, N-(6 Aminohexyl)-5-Chloro-2-Naphthalenesuflonamide, or N(6 Aminohexyl)-5-Bromo-2-Naphthalenesulfonamide in the Uterus to prevent Pregnancy” issued Dec. 6, 1983; U.S. Pat. No. 4,264,575 to Zimmerman et al., entitled “Contraceptive Methods and Compositions” issued Apr. 28, 1981; U.S. Pat. No. 5,698,524 to Mach et al., entitled “Method of treating a patient suffering from a viral infection” issued Dec. 16, 1997; U.S. Pat. No. 5,554,596 to Mach et al., entitled “Virus inhibiting composition” issued Sep. 10, 1996; U.S. Pat. No. 4,935,239 to Machida et al., supra; U.S. Pat. No. 3,914,433 to Ward et al., entitled “Method for Relieving the Discomforts of Pharyngitis” issued Oct. 21, 1975; U.S. Pat. No. 6,075,050 to Singh et al., entitled “Compound for inhibiting HIV infectivity” issued Jun. 13, 2000; and U.S. Pat. No. 4,185,097 to Ward et al., entitled “Method of combating Herpes simplex viruses with lignosulfonates” issued Jan. 22, 1980.

Similar biological properties are described for common sulfated polysaccharides, some of which affect sperm-egg interaction. For example, dextran sulfate can inhibit cell fusion. See, e.g., Krumbiegel M, et al., Dextran sulfate inhibits fusion of influenza virus and cells expressing influenza hemagglutinin with red blood cells (1992) Biochim Biophys Acta, 1110:158-164. In another example, dextran sulfate has a high affinity for mammalian sperm surface proteins, as described in Peterson R N, et al., Further characterization of boar sperm plasma membrane proteins with affinity for the porcine zona pellucida (1985) Gamete Res, 12:91-100. This association with sperm surface inhibits sperm-egg fusion in both the mouse and hamster and inhibits human sperm attachment to the zona pellucida. See, e.g., the following publications and references cited within: Ponce R H, et al., Inhibition of sperm-egg fusion in the hamster and mouse by carbohydrates (1994) Zygote, 2:253-262; Oehninger S, et al., Nature of the inhibitory effect of complex saccharide moieties on the tight binding of human spermatozoa to the human zona pellucida (1991) Fertil Steril 55:165-169. Similarly, fucoidan, a large polysulfated and negatively charged polysaccharide, can disrupt the organization of cell culture monolayers in a specific manner, as well as inhibit sperm-egg interaction in both sea urchins. See, e.g., Glabe C G, et al., Reversible disruption of cultured endothelial monolayers by sulphated fucans (1983) J Cell Sci, 61:475-490; Glabe C G, et al., Carbohydrate specificity of sea urchin sperm bindin: a cell surface lectin mediating sperm-egg adhesion (1982) J Cell Biol, 94:123-128; Deangelis P L, and Glabe C G. Polysaccharide structural features that are critical for the binding of sulfated fucans to bindin, the adhesive protein from sea urchin sperm (1987) J Biol Chem, 262:13946-13952; Deangelis P L, and Glabe C G. Role of basic amino acids in the interaction of binding with sulfated fucans (1988) Biochemistry, 27:8189-8194; and mammals. This is further described in Peterson R N, et al., Evidence for specific binding of uncapacitated boar spermatozoa to porcine zonae pellucidae in vitro (1984) J Exp Zool, 231:137-147; Oehninger S, et al., Antagonistic and agonistic properties of saccharide moieties in the hemizona assay (1990) Fertil Steril, 53; and, Oehninger S, et al., Effect of fucoidin on human sperm-zona pellucida interactions (1992) J Androl, 13:519-525.

The mechanism of bioactivity of sulfated macromolecular anions is complex, e.g., they can act on specific cell surface receptor conformation, rather than acting nonspecifically through charge-related effects. This is further described in the following publication and references cited within: Yoshida O, et al., Sulfation of the immunomodulating polysaccharide lentinan: a novel strategy for antivirals to human immunodeficiency virus (HIV) (1988) Biochem Pharmacol 37:2887-2891; Suzuki H, et al., Lignosulfonate A Water-Solubilized Lignin from the Waste Liquor of the Pulping Process Inhibits the Infectivity and Cytopathic Effects of Human Immunodeficiency Virus In-Vitro (1989c) Agricultural and Biological Chemistry, 53:3369-3372; Suzuki H, et al., Inhibition of the infectivity and cytopathic effect of human immunodeficiency virus by water-soluble lignin in an extract of the culture medium of Lentinus edodes mycelia (LEM). (1989b) Biochem Biophys Res Commun, 160:367-373; and, Miller D J, and Ax R L. Carbohydrates and fertilization in animals (1990) Mol Reprod Dev, 26:184-198.

Natural Products

It is also desirable to have natural contraceptives, i.e., those derived from natural sources. The present invention includes sulfonated compounds isolated and/or derived from natural sources. A variety of natural sources can provide the source of the sulfonated compounds of the present invention. For example, the natural source of the sulfonated compound can be a lignin, which is described in more detail below. Other natural sources of sulfonated compounds include, e.g., a plant, e.g., a fungus, e.g., an algae and the like.

Natural source compounds have the advantage in that they are isolated natural contraceptives and come from natural sources that are abundant and renewable in the world. In addition, natural source compounds can also act as microbicides. For example, see the following publications and references cited within: Zacharopoulos V R, and Phillips D M. Vaginal formulations of carrageenan protect mice from herpes simplex virus infection (1997b) Clin Diagn Lab Immunol, 4:465-468; and, U.S. Pat. No. 4,185,097 to Ward and Tankersley, supra.

Lignins and Derivatives

Compounds comprising a lignin and/or a derivative thereof can also be used in the methods, compositions and devices of the present invention to inhibit and/or interfere with fertilization. These compounds are non-cytotoxic and exhibit other beneficial activities, e.g., anti-virals. The lignin and/or the derivative thereof can also be source of sulfonated compounds. As mentioned above, lignin is a natural source for sulfonated compounds.

Lignin is abundant in nature. It is contained in almost all plants. Lignin can be isolated by a variety of methods and sources. In one example, lignin derivatives can be isolated by centering the spent liquor in the pulp and paper industry. This is described further in U.S. Pat. No. 4,935,239, as described above. In other examples, lignin can be isolated by extraction of wood, wood-like material and/or plant-cell cultures in an aqueous media under weakly acidic or alkaline conditions. Examples of wood-like material can include native wood, e.g., tree heartwood in the form of “milled wood”, softwood, hardwood, pomaceous fruit skin, nutshells, general woody plant constituents, grasses, e.g., esparto, wood analogues, e.g., woody substances produced by plant-cell cultures, synthetic wood analogues and the like. Alternatively, lignin can be isolated by extraction of wood-incarbonization products and bioconverted wood-like materials. Examples of wood incarbonization products or bioconverted wood-like materials include, e.g., native products of incarbonization, e.g., wood bioconverted by lignolytically active micro-organisms like white wood-putrefying fungi, e.g., wood bioconverted through the effect of isolated lignolytic enzymes and the like. These procedures are further described in U.S. Pat. No. 5,698,524 and U.S. Pat. No. 5,554,596, as described above.

Various compounds can be isolated from lignins. Typically, lignin-derived macromolecules (LDMs) are highly sulfonated and negatively charged compounds. For example, LSA can be isolated from lignins. LSA is a member of a family of related lignin-derived macromolecules (LDMs) that are byproducts, e.g., formed as a result of the conversion of wood pulp into paper. For example, LSA can be derived from the sulfite pulping process whereby wood chips are extracted with acidic aqueous sulfur dioxide, resulting in the depolymerization and dissolution of lignin, to produce a cellulose fiber (see, e.g., McCubbin (1983), supra). The aqueous effluent of this process can contain the polar breakdown product(s) of lignin, e.g., LSA. Another example of a LDM includes a sulfonated kraft lignin (or alkali lignin). A sulfonated kraft lignin can be derived from the kraft or sulfate pulping process.

LDMs are also non-cytotoxic. For example, LDMs inhibit fertilization in a number of non-mammalian species, without showing cytotoxicity. This is further described in the following publications and references cited within: Higashi R M, et al., A Polar High Molecular Mass Constituent of Bleached Kraft Mill Effluent Is Toxic To Marine Organisms (1992b) Environmental Science & Technology, 26:2413-2420; Cherr G N, et al., Electrophoretic separation, characterization, and quantification of biologically active lignin-derived macromolecules (1993) Anal Biochem, 214:521-527; and, Pillai M C, et al., Inhibition of the sea urchin sperm acrosome reaction by a lignin-derived macromolecule. (1997) Aquatic Toxicology (Amsterdam) 37:139-156. In another example, the oral toxicity of LSA is virtually non-toxic orally (LD50>40 g/kg) and has been used for many years as an animal feed additive due to its antipepsin activity and the protection it provides against the development of gastric ulcers. This is further described in the following publications and references cited within: Vocac J A, and Alphin R S, Effects and mechanism of action of a lignosulphonate on experimental gastric ulceration in rats (1968) Eur J Pharmacol, 4:99-102; Vocac J A, and Alphin R S, Antiulcerogenic and pepsin inhibitor properties of lignosulfonates (1969) Archives Internationales de Pharmacodynamie et de Therapie, 177:150-158; and, Luscombe D K, and Nicholls P J. Acute and subacute oral toxicity of AHR-2438B, a purified sodium lignosulphonate, in rats (1973) Food and Cosmetics Toxicology, 11:229-237.

LDMs exhibit a number of biological activities. For example, a lignin-derived macromolecule, e.g., LSA, activates macrophages and induces proliferation of bone marrow cells as described in Suzuki H, et al., Lignosulfonate from Waste Liquor of pulping Process Activates Murine Macrophages and Causes Proliferation of Bone Marrow Cells, (1989a) Agricultural and Biological Chemistry 53:1197-120. Other activities include potent inhibitors of the human immunodeficiency virus (HIV) in vitro, e.g., through interference with the CD-4 receptor/HIV interaction. This is further described in the following publications and references cited therein: Suzuki H, et al., (1989c), supra; and Suzuki H, et al., (1989b), supra. In vitro microbicidal activity of LDMs has been demonstrated with several other viruses as well. See, e.g., Sorimachi et al., Morphological changes in tumor cells caused by macrophages treated with lignin derivatives, (1990) Agric. Biol. Chem., 54:1611-1613. This anti-viral and anti-pathogen activity can be cell surface-mediated and can be related to the inhibitory effects of other sulfated molecules. See, e.g., Zaretzky F R, et al., Sulfated polyanions block Chlamydia trachomatis infection of cervix-derived human epithelia, (1995) Infect Immun, 63:3520-3526; Pearce-Pratt R, and Phillips D M. Sulfated polysaccharides inhibit lymphocyte-to-epithelial transmission of human immunodeficiency virus-1, (1996) Biol Reprod 54:173-182; Zacharopoulos V R, and Phillips D M. Cell-mediated HTLV-I infection of a cervix-derived epithelial cell line (1997a) Microb Pathog, V23:225-233.

LDM also exhibit specific cell type activities. For example, an LDM inhibits fibroblast growth and hepatocyte mitosis, yet it does not affect macrophages or carcinoma cells. See, e.g., Sorimachi K, et al. Inhibition of fibroblast growth by polyanions, effects of dextran sulfate and lignin derivatives (1992) Cell Biol Int Rep, 16:63-71. LDMs also inhibit fertilization in echinoderms without showing cytotoxic effects on other cells, see, e.g., Higashi R M, et al., (1992b), supra; and, Cherr et al., 1993, supra, via inhibition of the sperm acrosome reaction, as described in Pillai et al., 1997, supra.

### EXAMPLES

The following examples are offered to illustrate, but not to limit the claimed invention.

### Example 1

LSA Treated Sperm Prevents Sperm's Ability to Bind to the Zona Pellucida

This example involved the effect on LSA on sperm from adult male cynomologus macaques. Sperm was collected, mixed with LSA, washed and capacitated. The treated sperm was then used to determine its ability to bind to the zona pellucida or to induce fertilization.

The following chemicals were used. HPLC grade water was obtained from Fisher Scientific (Santa Clara, Calif.). Dulbecco's phosphate buffered saline (DPBS) and modified Biggers, Whitten, and Whittingham medium (Hepes-buffered BWW) were prepared by Irvine Scientific (Irvine, Calif.). CMRL 1066 medium was prepared by Gibco, heparin was obtained through Elkins-Sinn, Inc. (Cherry Hill, N.J., USA), fetal bovine serum (FBS) was provided by HyClone (Logan, Utah, USA), and buffalo rat liver (BRL) cells by American Type Culture Collection (Rockville, Md., USA). All recombinant hormones were supplied by Ares Serono (Randolph, Mass.). All other chemicals and salts for media preparation were purchased from Sigma Chemical Company (St. Louis, Mo.).

Lignosulfonic acid (LSA; Aldrich Chemical) was sequentially extracted with methylene chloride, and acetonitrile, e.g., as described in Higashi R M, et al., (199b), supra. The extracted LSA was extensively dialyzed (3.5 kDa cutoff) against distilled water to remove all salts and traces of solvent. The dialyzed LSA was then lyophilized in aliquots and stored at −20° C.

Four adult male cynomolgus macaques which were caged individually at the California Regional Primate Research Center (CRPRC) in compliance with the Federal Animal Welfare Act and the NIH Guidelines for Care and Use of Laboratory Animals. The animals were maintained on a 12L:12D light cycle at 25-27° C. and were given a diet of Purina monkey chow and water ad libitum. Semen samples were collected by electroejaculation, e.g., as described in Sarason et al., The use of nonmetal electrodes in ejaculation of restrained but unanesthetized macaques, (1991) J. Med. Primatol., 20:122-125, into 15 ml centrifuge tubes containing 5 ml of Hepes-buffered BWW maintained at room temperature (RT). After 15-30 min, the coagulum was removed, the semen samples were further diluted with an additional 5 ml of Hepes-buffered BWW containing 3 mg/ml bovine serum albumin (BSA) and then concentrated into a 1 ml pellet by centrifugation at 300×g for 10 minutes. LSA (previously dissolved in hepes-buffered BWW salts) was added to half of the pellet to give a final concentration of 1.5 mg LSA/ml of sperm. An equal volume of hepes-buffered BWW salts was added to the other half of the pellet. Both aliquots were incubated at room temperature for 40 minutes and then washed and capacitated, e.g., as described in Tollner T L, et al., Soybean trypsin inhibitor as a probe for the acrosome reaction in motile cynomolgus macaque sperm (2000) Zygote 8:127-137. Briefly, sperm were centrifuged through 3.5 ml of 80% Percoll for 25 minutes at 400 g. The supernatant containing Percoll and any remaining sperm was removed and the sperm pellet was resuspended in bicarbonate-buffered BWW, e.g., as described in Overstreet et al., Penetration of human spermatozoa into the human zona pellucida and the zona-free hamster egg: a study of fertile donors and infertile patients, (1980), Fertil. Steril., 33:534-542; and, Overstreet et al., In vitro capacitation of human spermatozoa after passage through a column of cervical mucus, (1980) Fert. Steril., 34:604-606, containing 30 mg/ml of BSA. Sperm were washed two more times by centrifugation at 300 g for 10 minutes and dilution in this medium. Sperm were finally resuspended at a concentration of 10-20×10⁶/ml in the bicarbonate-buffered BWW and were capacitated by a series of incubations beginning with a 24 hour incubation at room temperature in 4.5% CO₂. Following this room temperature incubation, sperm suspensions were incubated at 37° C. in 4.5% CO₂ for 2 more hours, at which point the sperm concentration was adjusted to 4×10⁶/ml for zona pellucida binding experiments or 500,000 motile sperm/ml for IVF. The sperm suspensions were activated by incubation for an additional hour in media containing 1 mM caffeine and 1 mM dbcAMP. About forty-five minutes following activation, an aliquot of control sperm for both IVF and the zona binding assay was treated with 1.5 mg/ml LSA for 15 minutes prior to introducing eggs into the sperm suspensions or addition of sperm to the zona pellucida binding chamber (see below). Following activation, 200 sperm per treatment were scored for percent motility at 200× magnification with phase-contrast optics using a BH-2 series Olympus microscope. Progression was assessed with a 0-4 rating system (0=no progressive sperm; 1=1-25% of motile sperm have forward (space gaining) progressive motility; 2=26-50% of motile sperm have forward progressive motility; 3=51-75% of motile sperm have forward progressive motility; 4=76-100% of motile sperm have forward progressive motility).

To determine sperm binding to zona pellucida, ovaries were obtained at necropsy from adult female cynomolgus macaques at the CRPRC. Zona pellucida-intact immature oocytes were collected from the ovaries and were frozen at −80° C. in 2 M dimethyl sulfoxide (DMSO) in DPBS according to previously published protocols, e.g., as described in VandeVoort et al., Sperm-zona pellucida interaction in cynomolgus and rhesus macaques, (1992) J. Androl. 13:428-432. The oocytes were thawed at 22° C. and rinsed through three dishes, each containing 0.5 ml of Hepes-buffered BWW medium, to remove DMSO prior to experiments. Oocytes were then deposited onto glass slides (one oocyte per slide) and allowed to air dry for over 10 minutes. Within 5 minutes prior to sperm zona coincubation, 2 μl of Hepes buffered BWW containing activator was added to the air-dried oocytes. Four post of silicon grease containing 50-75 μm beads were than deposited at four corners around the oocytes. A 22×22 mm glass coverslip was carefully pressed down onto the post until the grease was completely flattened. The slide was warmed on a microscope stage warmer set at 37.5° C. for 5 minutes prior to the addition of sperm. 40 μl of activated sperm preparation was added to the edge of the coverslip over the re-hydrated oocyte and was readily drawn by capillary action to fill the entire 22×22 mm space. Binding of sperm to the zona surface was observed with a Lietz Laborlux microscope with phase contrast optics at 400×. A timer was started at the moment the first motile sperm attached to the zona. Due to the depth of the preparation, sperm were restricted to bind to the outer edges of the zona. The total number of bound sperm was counted after 3 minutes starting at the 12 o'clock position of the oocyte and working clockwise to the starting point. The count required on average about 15 seconds to complete. Two oocytes (replicates) were used for each treatment for each male.

In a separate set of experiments, macaque sperm were treated with 1.5 mg/ml Fucoidan either prior to Percoll washing or after activation (as described for LSA above) and then were assessed for sperm-zona pellucida binding as described above. Fucoidin is a polysulfated compound shown to inhibit sperm-zona pellucida binding in several mammal species. This is further described in the following publications and references cited within: Mahoney, M C, et al., Fucoidin binding activity and its localization on human spermatozoa (1993) Contraception, 48:277-289; Peterson R N, et al., (1984), supra; Huang T T, and Yanagimachi R. Fucoidin inhibits attachment of guinea pig spermatozoa to the zona pellucida through binding to the inner acrosomal membrane and equatorial domains (1984) Exp Cell Res, 153:363-373; Oehninger S, et al., (1990), supra; Oehninger S, et al., (1991), supra; and, Oehninger S, Clark G F, et al., (1992), supra. The extent, orientation and distribution of the sulfation charge along anionic polymers of fucoidin are implicated as determining their relative affinities for a ligand. See, e.g., Deangelis P L, and Glabe C G. Specific recognition of sulfate esters by binding, a sperm adhesion protein from sea urchins (1990) Biochim Biophys Acta, 1037:100-105; and, Kim J G, et al., Charge-based interactions of mammalian sperm with oocytes: inhibition of fertilization of mouse oocytes by ligands of macrophage scavenger receptor(s) (1997) Fertil Steril, 68:1108-1113.

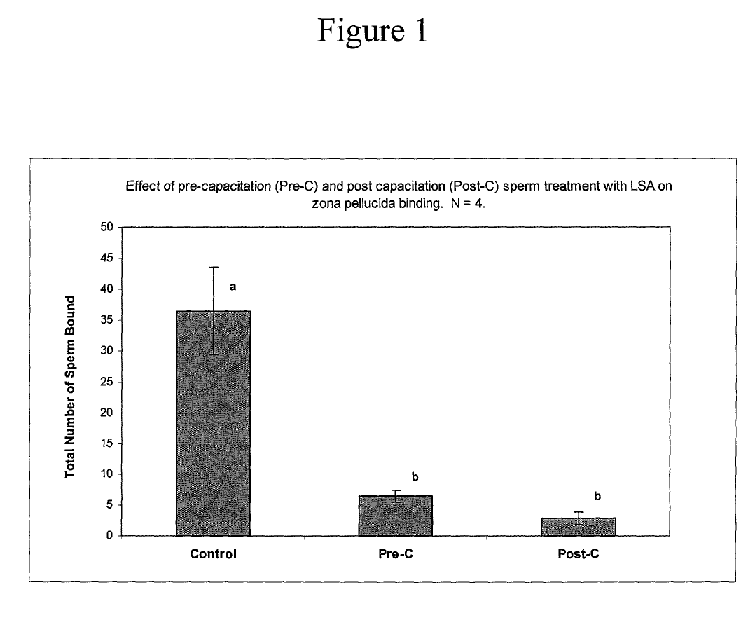

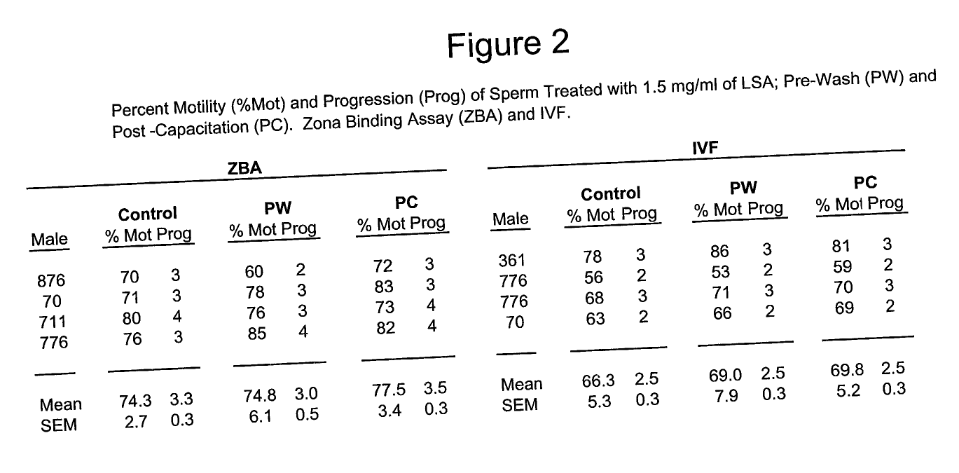

As shown in FIG. 1, LSA effects the ability of capacitated sperm to bind to the zona pellucida of immature oocytes. Specifically, post capacitation (PC or Post-C) addition of LSA to sperm inhibited binding on average by 92.5% (p<0.001; ANOVA at alpha=0.01 with pairwise comparisons using Tukey range test; SAS statistical software, SAS institute, Cary, N.C.) as compared to controls. Furthermore, LSA still inhibited sperm-zona binding by 82.5% (p<0.001) when added to sperm prior to washing through Percoll (pre-capacitation (Pre-C), also called pre-wash (PW)) and overnight incubation. The inhibitory effect of LSA on zona binding following pre- and post-capacitation treatments did not differ significantly although post-capacitation treatment consistently resulted in slightly greater inhibition. See FIG. 1. Neither the percentage of motile sperm or sperm progression were effected by LSA treatment. See FIG. 2. A high percentage of LSA-treated and control sperm (>75%) developed hyperactivated motility patterns consistent with capacitation. LSA-treated sperm appeared to strike the zona with equal frequency as the control sperm but were more likely to either bounce off immediately or pull themselves away after several seconds before a more “secure” attachment could be made.

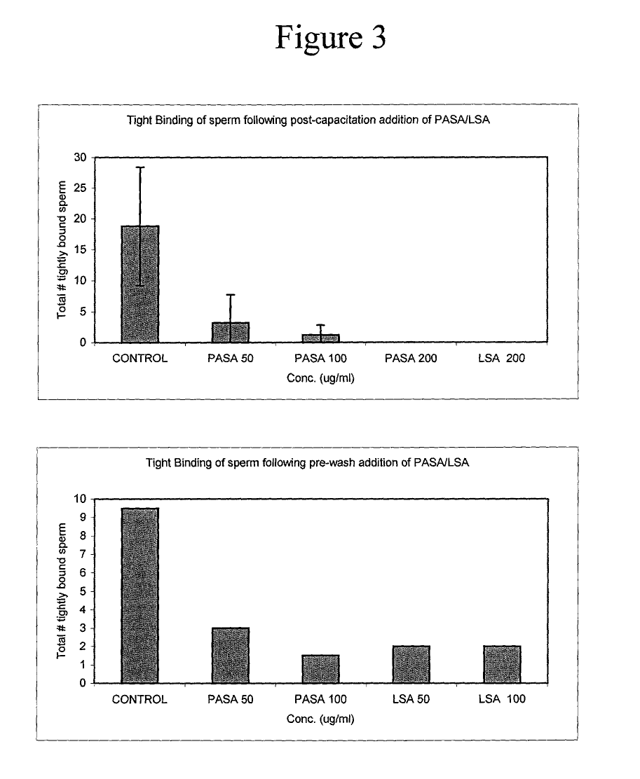

FIG. 3 shows zona pellucida binding experiments using sperm treated with PASA or LSA following post-capacitation (PC) or pre-wash (PW). As seen with LSA, PASA-treated sperm inhibited their binding to the zona pellucida of immature oocytes.

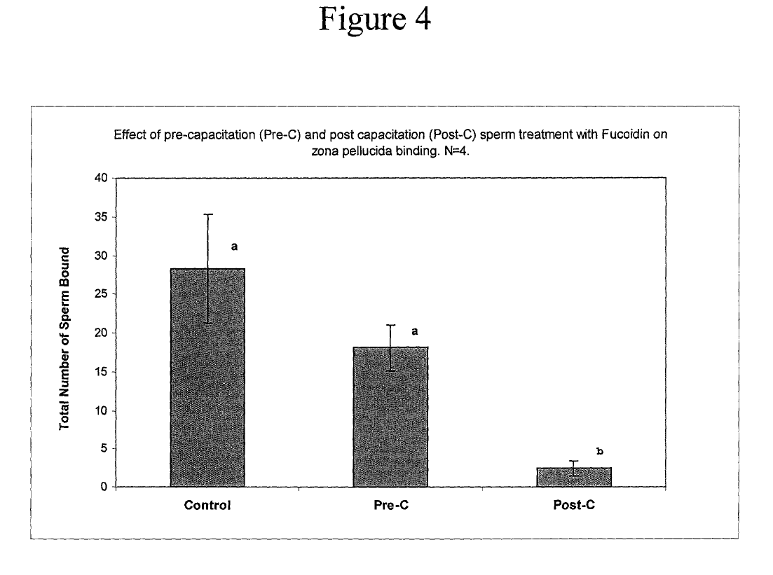

As mentioned above, zona pellucida binding experiments were repeated using Fucoidn. As with LSA, the effect of fucoidan on sperm was compared following treatment before (Pre-C) and after capacitation (PC or Post-C). Fucoidan significantly inhibited binding by 91.5% (p<0.001; ANOVA at alpha=0.01 with Tukey range testing) as compared to controls when the compound was added post-capacitation (PC or Post-C). See FIG. 4. A 36% inhibition of binding resulted from addition of fucoidan pre-capacitation, but this change was not significant from controls. See FIG. 4. Fucoidin was effective to inhibit zona binding when present during coincubation with gametes, but not when sperm were treated prior to capacitation. No motility changes were observed with addition of fucoidan either pre- or post-capacitation. The reason for the apparent difference in avidity for the sperm surface of the two macromolecules can lie in the differences in the nature of the sulfur moiety, e.g., sulfate (fucoidan) or sulfonate (LSA), and/or can lie in the differences in the concentration and orientation of anionic charge. For example, LSA has a complex, highly branched structure that can promote “binding” to a large number of sperm surface receptors. Since non-capacitated sperm with bound LSA results in a block to zona binding, some form of a zona receptor(s) can be exposed in fresh sperm, although, some sperm receptors for zona ligands may not be exposed until after capacitation. See, e.g. Iborra et al., Human sperm coating antigen from seminal plasma origin (1996) Am. J. Reprod. Immunol., 36:118-125; Youssef et al., Effect of sperm viability, plasmalemma integrity, and capacitation on patterns of expression of mannose-binding sites on human sperm, (1997), Arch. Androl., 38:67-74; and, Fraser, Sperm capacitation and the acrosome reaction (1998) Hum Reprod. 13 Suppl 1:9-19.

### Example 2

LSA Prevents In Vitro Fertilization

In this example, in vitro fertilization of four female cynomolgus macaques was examined using treated sperm as described above in Example 1. Chemicals, animals and preparations were the same as in Example 1, except where indicated below.

To determine in vitro fertilization, four female cynomolgus macaques, maintained at CRPRC as described above, were superovulated using injections of recombinant human gonadotropins. Beginning on the morning of day 1 to 4 of menses, females received FSH injections twice daily (30 IU) through treatment days 1-6 and FSH/LH (30 IU each) through treatment days 7-9. Females also received concurrent treatment with Antide® (1.0 mg/kg sc, once daily), a GnRH antagonist. All injections were administered in less than 0.4 ml total volume. Animals were anesthetized using ketamine (10 mg/kg) and were evaluated by ultrasounded for follicular development on treatment day 7. Animals with poor follicular development (follicular size<4 mm) were dropped from the study. When follicles >4 mm were detected, females received recombinant human chorionic gonadotropin (hCG, 40 IU intramuscular) the next morning and twenty-seven hours after the administration of hCG, follicles were aspirated at laparoscopy, e.g., as described in Zelinski-Wooten et al., Initiation of periovulatory events in gonadotrophin-stimulated macaques with varying doses of recombinant human chorionic gonadotropin, (1997) Hum Reprod. 13:554-560. On treatment day 11, females were anesthetized using ketamine and placed under dorsal recumbency with isoflurane inhalation anesthesia for laparoscopic oocyte retrieval. A 1 cm midventral incision was made into the skin and fascia and a Verres needle was introduced into the abdominal cavity. Carbon dioxide was used to insufflate the abdomen to approximately 12 mmHg pressure. A 5 mm trocar was then introduced into the same incision and the 5 mm laparoscope was introduced. A 1 cm incision was placed into the right caudal abdomen and an additional 5 mm port was introduced for grasping forceps. Each ovary was suspended sequentially for oocyte retrieval by atraumatic forceps at the ovarian proper ligament. A 3 inch 22 gauge needle attached to mild vacuum pressure (50-60 mmHg) was introduced into the abdomen and each visible follicle was then aspirated into a 15 ml sterile tissue culture tube containing 5 ml TALP medium. The instruments were then withdrawn and each incision was closed using standard procedures. The females were recovered and given oxymorphone (0.15 mg/kg, im) for postoperative pain for 1-2 days. The pooled aspirates from left and right ovaries were placed in TH3 medium and immediately transported to the laboratory for recovery of oocytes.

Cumulus-oocyte complexes (COC) were aspirated from follicles and placed in TH3 medium, e.g., as described in Nusser et al., Developmental competence of oocytes after ICSA in the rhesus monkey, (2001), Human reproduction, 16: 130-137, containing 50 units/ml heparin at 37° C. COCs or oocytes without cumulus were then identified and graded as to their maturation status under dissecting microscope. Mature oocytes (at Metaphase II stage with first polar body) with or without cumulus layers were washed in warm and equilibrated CMRL 1066 medium containing 10% fetal bovine serum, 10 mM L-glutamine, 5 mM sodium pyrumate, 1 mM sodium lactate, 100 units/ml penicillin and 100 μg/ml streptomycin, e.g., as described in Nusser et al., supra, for three times and kept in this medium in CO₂ incubator at 37° C. prior to fertilization.

Oocytes were incubated in the insemination drops covered with oil for 4 hours, and then the oocytes were washed in CMRL 1066 medium for three times, and cultured for 4-7 days at 37° C. in 5% CO₂ on buffalo rat liver (BRL) cells (American Type Culture Collection, Rockville, Md., USA)) in CMRL medium supplemented with 10% FBS, e.g., as described in Nusser et al., supra. 4-5 oocytes were used for each treatment for each IVF cycle. Oocytes containing two pronuclei and two polar bodies (12-16 hour post-insemination) and then cleaved into two cells (24-30 hour post-insemination) were considered fertilized and maintained in culture. Embryos were transferred to fresh plates of BRL cells every other day.

During the fourth IVF cycle, two-three eggs were removed from each insemination drop after the four hour coincubation period for observation of sperm on the zona pellucida. Eggs were lightly rinsed once and placed in fresh drop of CMRL 1066 maintained under oil in a clear culture dish. Eggs were photographed at 200× with an AxioCam digital camera (Carl Zeiss Vision GmbH, Germany) installed on an inverted Olympus Ix70 microscope with Hoffman modulation contrast (HMC) optics.

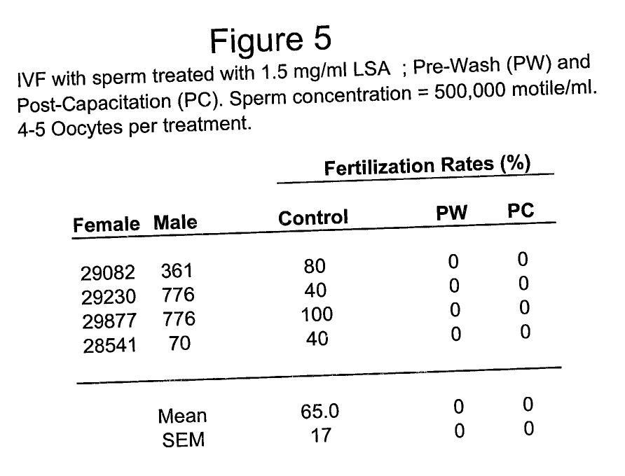

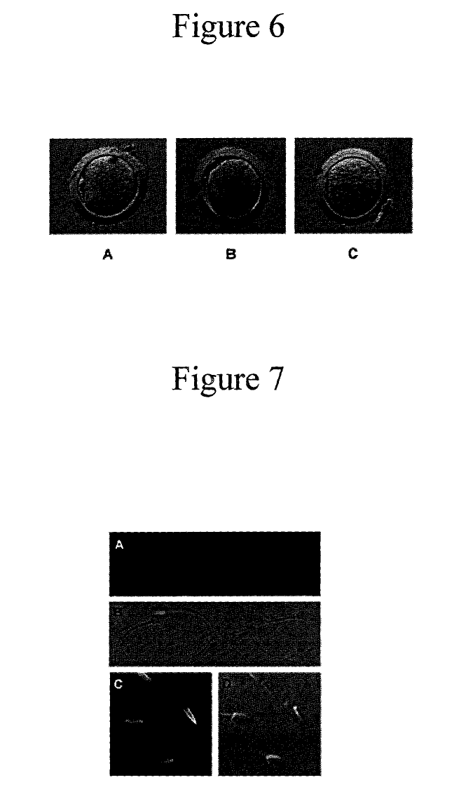

The results are shown in FIG. 5. As seen in FIG. 5, both pre-(prewash (PW) or Pre-C) and post-capacitation (Post-C or PC) treatment with LSA blocked fertilization in IVF trials. Only oocytes that were inseminated with control sperm formed pronuclei and underwent cleavage. As with the zona-binding studies, there was no difference between treatments on sperm motility. See FIG. 2. For two males, there was a drop in percent motility following dilution and activation which can account for the lower fertilization rates in the control groups. See FIG. 2. A difference in binding was apparent between oocytes incubated with control sperm and those incubated with LSA-treated sperm. See FIG. 6. FIG. 6 illustrates oocytes imseminated with control sperm, Panel A, sperm treated with LSA pre-capacitation, Panel B, and sperm treated with LSA post-capacitation, Panel C, following about a 4 hour coincubation of gametes for IVF. The oocytes were lightly rinsed once and photographed with an AxioCam digital camera (Carl Zeiss Vision GmbH, Germany) installed on an inverted Olympus Ix70 microscope with Hoffman modulation contrast optics. As a result, when ejaculated, non-capacitated macaque sperm are treated with LSA, they remain motile but are rendered infertile. This anti-fertility effect is maintained even after hours of capacitation and activation.

Example 3

Labeling of Sperm with LSA

In this example, localization of LSA to the sperm surface was determined using LSA conjugated with 5-(((2-carbhydrazino)methyl)thio)acetyl)-aminofluorescein (FITC-LSA; Molecular Probes, Eugene, Oreg.). Chemicals, animals and preparations were the same as in Example 1, except where indicated below.

The conjugate was prepared by conincubating 10 mg/ml purified LSA with 0.5 mg/ml of the fluorescent probe in PBS, pH 7.4 for 1 hour. Unreacted probe was removed by dialysis (2K MWCO Slide-A-Lyzer, Pierce Chemical Co., Rockford, Ill.) against 5% n-butanol in PBS then ddH₂O. Dialysis retentate (FITC-LSA) was lyophylized and stored dessicated at −20° C. until used.

Sperm were labeled both pre- and post capacitation with FITC-LSA, as described above. Prior to centrifugation through Percoll, an aliquot of labeled sperm was resuspended in DPBS and centrifuged again at 300×g for 10 minutes. The resulting pellet was fixed with 0.8% paraformaldehyde for 15 minutes. Fixed sperm were resuspended in DPBS and centrifuged again at 300×g for 10 minutes and observed for fluorescence. Sperm labeled with FITC-LSA pre-capacitation were observed for fluorescence following capacitation as were sperm treated with FITC-LSA post-capacitation. Sperm were fixed and washed in the same manner. FITC-LSA labeled sperm were observed with a Lietz Laborlux S microscope equipped with 200W mercury fluorescence vertical illuminator and a 1-Lamda Ploemopac incident light fluorescence illuminator employing an 13 filter cube with a BP 450-490 excitation filter, a RKP 0510 dichromatic mirror, and a LP 515 suppression filter.

Imaging of fluorescent sperm for the production of micrographs was performed as described in e.g., Yudin et al., (1998) “Rearrangement of the PH-20 protein on the surface of macaque spermatoza following exposure to anti-PH-20 antibodies or binding to zona pellucida” Mol. Reprod. Dev 50:207-220. Sperm were viewed with an Olympus upright BH-2 microscope (Scientific Instruments, Sunnyvale, Calif.) using a 60× oil immersion objective. The microscope was equipped with a Bio-Rad MRC-600 Laser scanning confocal system including a 15 mW krypton-argon mixed gas multiline laser (Bio-Rad, Hercules, Calif.). Sperm were objectively sections (0.25 m) and the full Z-series of images were collected and projected in order to determine surface labeling patterns. Images were digitally converted with Adobe Photoshop (Adobe Systems, San Jose, Calif.) and printed using dye sublimation.

The results of sperm labeled with FITC-LSA both pre- and post-capacitation are shown in FIG. 7. Pre-capacitation labeled sperm prior to Percoll separation fluoresced evenly over the entire surface of the head and tail. See FIG. 7, Panels A and B. Labeling pattern was observed in 100% of cells. When these sperm were washed through 80% Percoll and cacpacitation medium, the surface fluorescence was no longer detectable. To confirm that LSA maintains anti-fertility effects on sperm following conjugation to FITC, sperm treated with FITC-LSA prior to capacitation were assessed for ability to bind to the zona pellucida. FITC-LSA significantly inhibited binding compared to controls (1.3+/−1.1 sperm/zona verses 10.5+/−4.7 sperm/zona; mean +/− sem; p<0.01). Post-capacitation labeling with FITC-LSA resulted in bright labeling over the entire head in 80% of sperm with little or no labeling of the flagellum. See FIG. 7, Panels C and D.

Following capacitation, LSA binds primarily to the head in most sperm, and based on the increase in fluorescence intensity, can bind in greater quantities than on pre-capacitated sperm. Fluorescence is undetected in pre-capacitation FITC-LSA labeled sperm following washing. The label may be removed by Percoll. This can be due to the heterologous composition of LSA which can result in varying amounts of conjugated FITC to be detected by fluorescence and/or can be due to LSA induced changes on the sperm surface that are irreversible such that once it is removed, the antifertility effects still remain.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method for inhibiting fertilization, the method comprising administering an effective amount of a compound to an animal, the compound comprising at least one sulfonated compound, wherein the compound interacts with sperm and wherein the at least one sulfonated compound is selected from the group consisting of: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, and derivatives thereof.

2. The method of claim 1, wherein the interaction occurs at surface of the sperm.

3. The method of claim 1, wherein the interaction occurs at head of the sperm.

4. The method of claim 1, wherein the interaction is the at least one sulfonated compound binding to the sperm.

5. The method of claim 1, wherein the compound is a polysulfonated compound.

6. The method of claim 1, wherein the compound is in an aqueous solution.

7. The method of claim 1, wherein the compound inhibits the sperm interaction to a zona pellucida.

8. The method of claim 1, further comprising treating the sperm with the compound for at least about 3 minutes.

9. The method of claim 8, wherein the treatment occurs at about room temperature.

10. The method of claim 8, wherein the treatment occurs at about 37° C.

11. The method of claim 1, wherein the compound is administered after ejaculation.

12. The method of claim 1, wherein the compound is administered prior to ejaculation.

13. The method of claim 1, wherein the compound is administered vaginally.

14. The method of claim 1, wherein the compound is in a formulation, wherein the formulation is selected from the group consisting of a foam, a cream, a gel, a jelly, a douche, an aerosol, a film, a tablet, a sponge, a vaginal suppository, an impregnated tampon, a controlled delivery device, a vaginal ring, an intrauterine device, a lubricant on a male condom, a lubricant on a female condom, a lubricant on a cervical cap and a lubricant on a cap diaphragm.

15. The method of claim 1, wherein the animal is a primate.

16. A method for inhibiting fertilization, the method comprising administering an effective amount a compound derived from a natural source to an animal, the compound comprising at least one sulfonated compound, wherein the at least one sulfonated compound interacts with sperm.

17. The method of claim 16, wherein the at least one sulfonated compound is a polysulfonated compound.

18. The method of claim 16, wherein the at least one sulfonated compound is a lignosulfonic acid (LSA).

19. The method of claim 16, wherein the natural source is a lignin.

20. The method of claim 16, wherein the natural source is a plant, a fungus or an algae.

21. The method of claim 16, where the interaction occurs at surface of the sperm.

22. The method of claim 16, wherein the interaction occurs at head of the sperm.

23. The method of claim 16, wherein the interaction is the at least one sulfonated compound binding to the sperm.

24. The method of claim 16, wherein the compound inhibits the sperm interaction to a zona pellucida.

25. The method of claim 16, further comprising treating the sperm with the compound for at least about 3 minutes.

26. The method of claim 25, wherein the treatment occurs at about room temperature.

27. The method of claim 25, wherein the treatment occurs at about 37° C.

28. The method of claim 16, wherein the compound is administered after ejaculation.

29. The method of claim 16, wherein the compound is administered prior to ejaculation.

30. The method of claim 16, wherein the compound is administered vaginally.

31. The method of claim 16, wherein the compound is in a formulation, wherein the formulation is selected from the group consisting of a foam, a cream, a gel, a jelly, a douche, an aerosol, a film, a tablet, a vaginal suppository, a sponge, an impregnated tampon, a controlled delivery device, a vaginal ring, an intrauterine device, a lubricant on a male condom, a lubricant on a female condom, a lubricant on a cervical cap and a lubricant on a cap diaphragm.

32. The method of claim 16, wherein the animal is a primate.

33. A method for inhibiting fertilization, the method comprising administering an effective amount of a compound to an animal, the compound comprising at least one lignin or a derivative thereof, wherein the compound interacts with sperm.

34. The method of claim 33, wherein the derivative is sulfated.

35. A composition comprising at least one sulfonated compound, a pharmaceutically acceptable excipient and a sperm, wherein the at least one sulfonated compound is selected from the group consisting of: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, and derivatives thereof.

36. The composition of claim 35, wherein the compound is a polysulfonated compound.

37. A composition comprising at least one sulfonated compound and a spermicide, wherein the at least one sulfonated compound is selected from the group consisting of: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, and derivatives thereof.

38. A contraceptive device comprising a device and at least one sulfonated compound in a formulation, wherein the device is selected from one or more of: a sponge, a tampon, an intrauterine device, a vagina ring, a male condom, a female condom, a cervical cap or a diaphragm, wherein the formulation is selected from the group consisting of a foam, a cream, a gel, a jelly, a douche, an aerosol, a film, a suppository and a tablet, and wherein the at least one sulfonated compound is selected from the group consisting of: a lignosulfonic acid (LSA), a polyanetholesulfonic acid (PASA), a polyvinylsulfonic acid, a poly(2-acrylamido-2-methyl-1-propanesulfonic acid), a poly(2-acrylamido-2-methyl-1-propanesulfonic acid co-acrylonitrile), a poly(2-acrylamido-2-methyl-1-propanesulfonic acid-co-styrene), a poly(4-vinylpyridinium p-toluenesulfonate), a sulfonic acid azo dye, a sulfonic acid derivative of a porphyrin, a sulfonic acid derivative of a triphenylmethane, a sulfonic acid derivative of a stilbene, a sulfonated phenylpropane, a sulfonated kraft lignin, and derivatives thereof.

39. A composition comprising a compound isolated from a natural source, a pharmaceutically acceptable excipient and a sperm, wherein the compound interacts with the sperm and wherein the compound comprises at least one sulfonated compound.

40. The composition of claim 39, wherein the at least one sulfonated compound is a lignosulfonic acid (LSA).

41. The composition of claim 39, wherein the natural source is a lignin.

42. The composition of claim 39, wherein the natural source is a plant, a fungus or an algae.

43. A composition comprising a compound isolated from a natural source and a spermicide, wherein the compound comprises at least one sulfonated compound.

44. The composition of claim 43, wherein the at least one sulfonated compound is a lignosulfonic acid (LSA).

45. A contraceptive device comprising a device and a compound isolated from a natural source, the compound comprising at least one sulfonated compound in a formulation, wherein the device is selected from one or more of: a sponge, a tampon, an intrauterine device, a vagina ring, a male condom, a female condom, a cervical cap or a diaphragm and wherein the formulation is selected from the group consisting of a foam, a cream, a gel, a jelly, a douche, an aerosol, a film, a suppository and a tablet.

46. The contraceptive device of claim 45, wherein the sulfonated compound is a lignosulfonic acid (LSA).

47. The contraceptive device of claim 45, wherein the natural source is a plant, a fungus or an algae.

48. The contraceptive device of claim 45, wherein the natural source is a lignin.

49. A composition comprising a lignin or a derivative thereof, a pharmaceutically acceptable excipient and a sperm.

50. The composition of claim 49, wherein the derivative is sulfated.

51. A composition comprising a lignin or a derivative thereof and a spermicide.

52. The composition of claim 51, wherein the derivative is sulfated.

53. A contraceptive device comprising a device and a compound comprising at least one lignin or a derivative thereof in a formulation, wherein the device is selected from one or more of: a sponge, a tampon, an intrauterine device, a vagina ring, a male condom, a female condom, a cervical cap or a diaphragm and wherein the formulation is selected from the group consisting of a foam, a cream, a gel, a jelly, a douche, an aerosol, a film, a suppository and a tablet.

54. The contraceptive device of claim 53, wherein the derivative is sulfated.

55. A method of inhibiting fertilization, the method comprising administering an effective amount of a compound to an animal, the compound comprising at least one sulfonated compound, wherein the compound interacts with sperm and wherein the at least one sulfonated compound is other than a polystyrene sulfonate, a long chain alkyl sulfonate, a long chain alkenyl sulfonate, a sulfoalkyl alkanoate salt, a sodium tetradecyl sulfonate, a sulfonated hesperidin, a substituted benzenesulfonic acid formaldehyde co-polymer, a H₂SO₄-modified mandelic acid, a condensation polymer product produced a condensation reaction of an aromatic sulfonic acid and an aldehyde, a formaldehyde naphthalenesulfonic acid condensation polymer, a 8-anilino-1-naphthalenesulfonate, a N-(6 aminohexyl)-5-chloro-1-naphthalenesulfonamide, a N-(6 aminohexyl)-5-chloro-2-naphthalenesulfonamide, and a N-(6 aminohexyl)-5-bromo-2-naphthalenesulfonamide.
